(19) United States
(12) Patent Application Publication
(10) Pub. No.: US 2021/0080435 A1
(43) Pub. Date: Mar. 18, 2021

(54) DEVICES AND METHODS FOR FLAVOR ANALYSIS

(71) Applicant: EPC Natural Products Co., Ltd., Beijing (CN)

(72) Inventors: Jingang SHI, Beijing (CN); Haixia REN, Beijing (CN)

(21) Appl. No.: 17/018,086

(22) Filed: Sep. 11, 2020

Related U.S. Application Data
(60) Provisional application No. 62/902,035, filed on Sep. 18, 2019.

(51) Int. Cl. G01N 30/72; H01J 49/04; G01N 33/14

(52) U.S. Cl. CPC G01N 30/724; G01N 33/14; H01J 49/0445

(57) ABSTRACT

A sample testing device comprises an aerosol generator for generating aerosol from a test sample, a sample collector for collecting aerosol particles within a desired size range, and a sample analyzer for determining chemical composition of the collected aerosol particles. The sample testing device may be used to conduct an integrated orthonasal/retronasal flavor analysis for a food product or beverage.

### CROSS REFERENCE

This application claims the benefit of U.S. Provisional Patent Application No. 62/902,035, filed Sep. 18, 2019. The disclosure set forth in the referenced application is incorporated herein by reference in its entirety.

### BACKGROUND

Before products make their way onto grocery store shelves, they are tasted, touched, and smelled by sensory evaluators first. In spite of the fact that these sensory evaluators are paid professionals, their practice may be considered more of an art than a science. This is largely the case considering the complexities and uncertainties associated with olfactory/taste evaluations. Further, the subjective aspects of this art call into question the objectivity and legitimacy of this art or science, particular when viewed in the context of our understanding about sensory perception.

Many studies have reported that subjective taste intensity is enhanced by odors which are congruent, for example a sweet taste and a vanilla odor. Some reports have suggested that subjective taste is more strongly enhanced by retronasal than by orthonasal odors; others have suggested that taste enhancements by both odor routes are identical. Differences between the two routes include the direction of airflow accompanying breath.

In view of the complexities associated with taste and olfactory integration, there is a need for developing more accurate means for evaluating consumable products in view of the current understanding.

### SUMMARY

One aspect of the present application is directed to a sample testing device comprising: an aerosol generator for generating aerosol from a test sample, a sample collector for collecting aerosol particles within a desired size range, and a sample analyzer for determining chemical composition of the collected aerosol particles.

In some embodiments, the testing device further comprises a first port for delivering the collected aerosol particles to a retronasal passageway of a subject; and a second port for delivering the collected aerosol particles to the sample analyzer. In some embodiments, the aerosol generator comprises an atomizer.

Another aspect of the present application is directed to a method for evaluating and analyzing flavor. The method comprises the steps of generating aerosol sample from a test sample, collecting the aerosol sample, evaluating the flavor of the collected aerosol sample; and detecting the chemical composition of the collected aerosol sample.

In some embodiments, the method further comprises the step of determining the size distribution and concentration of the particles in the aerosol sample. In some embodiments, the method further comprises the step of pretreating the aerosol sample prior to the detecting step.

Another aspect of the present application is directed to a method for identifying chemical species associated with an olfactory taste attribute. The method comprises the steps of

(a) generating a first aerosol sample from a first test sample;

(b) subjecting a portion of the first aerosol sample to the retronasal and/or passageway of the subject and generate a first retronasal and/or orthonasal taste profile;

(c) delivering another portion of the first aerosol sample to a sample analysis device, and generating a chemical fingerprint of the first aerosol sample;

(d) generating a second aerosol sample from a second test sample;

(e) subjecting a portion of the second aerosol sample to the retronasal and/or passageway of the subject and generate a second retronasal and/or orthonasal taste profile;

(f) delivering another portion of the second aerosol sample to a sample analysis device, and generating a chemical fingerprint of the second aerosol sample; and

(g) correlating the first and second taste profiles with the first and second chemical fingerprint to identify one or more chemical species associated with an olfactory taste attribute present in the taste profiles.

### BRIEF DESCRIPTION OF THE DRAWINGS

FIG. 1 is a block diagram of an exemplary sample testing device 100 for retronasal and orthonasal flavor testing and analysis.

FIG. 2 is a flow chart depicting a method for flavor testing and analysis.

FIG. 3 is another flow chart showing another embodiment of the method of the present application.

FIG. 4 is a diagram showing an embodiment of the testing device of the present application.

FIG. 5 is a diagram showing another embodiment of the testing device of the present application.

FIG. 6 is a diagram showing another embodiment of the testing device of the present application.

FIG. 7 is a diagram showing an embodiment of the testing device of the present application.

### DETAILED DESCRIPTION

### I. Definitions

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably. Further, any reactant concentrations described herein should be considered as being described on a weight to weight (w/w) basis, unless otherwise specified to the contrary (e.g., mole to mole, weight to volume (w/v), etc.)

The term “volatile”, as used herein, refers to a compound having a measurable vapor pressure at room temperature, and/or exhibits a vapor pressure of, or greater than, about 2 mm. of mercury at 20° C.

The term “non-volatile”, as used herein, refers to a compound having a negligible vapor pressure at room temperature, and/or exhibits a vapor pressure of less than about 2 mm. of mercury at 20° C.

The terms “flavor” and “flavor characteristic” are used interchangeably with reference to the combined sensory perception of one or more components of taste, odor, and/or texture.

The terms “flavoring agent”, “flavoring” and “flavorant” are used interchangeably with reference to a product added to food or beverage products to impart, modify, or enhance the flavor of food. As used herein, these terms do not include substances having an exclusively sweet, sour, or salty taste (e.g., sugar, vinegar, and table salt).

The term “natural flavoring substance” refers to a flavoring substance obtained by physical processes that may result in unavoidable but unintentional changes in the chemical structure of the components of the flavoring (e.g., distillation and solvent extraction), or by enzymatic or microbiological processes, from material of plant or animal origin.

The term “synthetic flavoring substance” refers to a flavoring substance formed by chemical synthesis.

The term “enhance,” as used herein, includes augmenting, intensifying, accentuating, magnifying, and potentiating the sensory perception of a flavor characteristic without changing the nature or quality thereof.

Unless otherwise specified, the terms “modify” or “modified” as used herein, includes altering, varying, suppressing, depressing, fortifying and supplementing the sensory perception of a flavor characteristic where the quality or duration of such characteristic was deficient.

The phrase “sensory profile” or “taste profile” is defined as the temporal profile of all basic tastes of a sweetener. The onset and decay of sweetness when a sweetener is consumed, as perceived by trained human tasters and measured in seconds from first contact with a taster's tongue (“onset”) to a cutoff point (typically 180 seconds after onset), is called the “temporal profile of sweetness”. A plurality of such human tasters is called a “sensory panel”. In addition to sweetness, sensory panels can also judge the temporal profile of the other “basic tastes”: bitterness, saltiness, sourness, piquance (aka spiciness), and umami (aka savoriness or meatiness). The onset and decay of bitterness when a sweetener is consumed, as perceived by trained human tasters and measured in seconds from first perceived taste to the last perceived aftertaste at the cutoff point, is called the “temporal profile of bitterness”.

The phrase “sucrose equivalence” or “SE” is the amount of non-sucrose sweetener required to provide the sweetness of a given percentage of sucrose in the same food, beverage, or solution. For instance, a non-diet soft drink typically contains 12 grams of sucrose per 100 ml of water, i.e., 12% sucrose. This means that to be commercially accepted, diet soft drinks must generally have the same sweetness as a 12% sucrose soft drink, i.e., a diet soft drink must have a 12% SE. Soft drink dispensing equipment assumes an SE of 12%, since such equipment is set up for use with sucrose-based syrups.

As used herein, the term “off-taste” refers to an amount or degree of taste that is not characteristically or usually found in a beverage product or a consumable product of the present disclosure. For example, an off-taste is an undesirable taste of a sweetened consumable to consumers, such as, a bitter taste, a licorice-like taste, a metallic taste, an aversive taste, an astringent taste, a delayed sweetness onset, a lingering sweet aftertaste, and the like, etc.

### II. Method and Integrated System for Flavor Evaluation and Analysis

Fossils of mammals and their extinct relatives among cynodonts give evidence of correlated transformation affecting olfaction as well as mastication, head movement, and ventilation, and suggest evolutionary coupling of these seemingly separate anatomical regions into a larger integrated system of ortho-retronasal olfaction. Evidence from paleontology and physiology suggests that ortho-retronasal olfaction played a critical role for mammalian cortical evolution. Human cortical evolution was enhanced by ortho-retronasal smell.

The world of taste and flavor is extraordinary complicated, not only in terms of the senses involved but also in relation to the words used to describe it. Words such as “taste” and “flavor” are interchangeably used in our daily lives without giving too much thought to their precise meaning to describe the sensory impressions of food and beverage inaccurately.

Currently, “taste” scientifically is the term to describe the sensation recognition of substances by the taste buds on the tongue in the oral cavity. Flavor, odor, smell and aroma are used loosely to describe the perceived sensation through the olfactory system. Odor and smell are generally used for description by nose sniffing-in via orthonasal olfactory. Aroma as a positive word usually is used to describe the pleasant smell. Flavor scientifically is defined as volatile substances but often is used to describe overall flavor impression of taste, smell, and touch. Mouthfeel by its name is used to describe the physical stimuli including pain, temperature, and tactile sensation such as pressure, touch, stretching and vibrations in the oral cavity or mouth. To summarize our daily used terms, tongue takes full credit for the taste which is related to food and beverage ingredients such as acids and sweeteners. Oral cavity takes all credit for mouthfeel, which is related to food and beverage ingredients as texture such as fiber and thickeners. Volatile substances via Orthonasal olfactory sniffing-in and retronasal olfactory breathing-out takes the credit for the smell and aroma. The brain combines input from taste, smell, and other senses to create the multi-modal of overall sensation of flavor.

Recently, scientists from the Monell Center report that functional olfactory receptors, the sensors that detect odors in the nose, are also present in human taste cells found on the tongue. G-protein coupling receptors detecting the taste also exist in nasal epithelium, but claimed to be the odor receptors. It is still mysterious about the mechanism how the general image of food and beverage perceived by human being.

The general understanding is: Once the food is in the mouth, mechanical handling of the food takes over, while the salivary juices start to flow. If the food is liquid, it might be swirled around in the mouth a few times before it is swallowed. If the food is quite firm, the chewing movement of the jaws and the teeth will reduce it to small pieces, while the tongue is carrying out acrobatic maneuvers that mix everything together. At the same time, more taste substances are released and dissolved in the saliva, diffuse in the oral cavity, and are picked up by the taste buds, while volatile substances drift up into the nasal cavity and activate the odor receptors, especially when we exhale. In case the beverage is in the mouth, the liquid is distributed in the oral cavity, and taste substances dissolved in the water are sensed by taste buds, and volatile substances are elevated into retronasal olfactory system once swallowed.

Scientifically, it is known that brain creates the general impression of flavor, the retronasal olfactory should take most of the credit. There are interaction between smell and taste, mouthfeel and taste, smell and taste. However, it is still unclear what initiate these interaction, there are a lot of sensory confusion among the taste, mouthfeel and smell, which is not simply resulted by the fact that we have difficulty in distinguishing these three senses, but more due to profound neurological reasons that brain plays the tricks and lead us to aggregate completely discrete sensory impression into one integrated impression or a single memory.

In additional to its function in smell perception, olfactory cortex plays a role of sensor to amino acids in the diet. Evidence from Dorothy Gietzen indicates that the pyramidal cells contain a molecular mechanism that senses the lack of an essential amino acids molecules with the amino acids. However, how this is communicated to the cell membrane to charge the cell's activity, and what is the pathway for communicating this message to the rest of the brain are still unclear, because theoretically, amino acids are non-volatile substances, it could not reach the olfactory bulbs when eating. Therefore, there is a need to develop a new theory, a new method, a new device to measure the link between food and beverage to the overall function of olfactory cortex.

The inventor hypothesized that the overall flavor is a casted image created by brain, it should be not simply assembled by different senses. Taste, mouthfeel and flavor are simultaneously by gustatory, olfactory and somatosensory system. Overall flavor is more than the sum of its parts.

Unlike conventional thinking:

Flavor=taste in tongue+mouthfeel+smell (sniffing-in)

The overall flavor should be:

Flavor=Gustatory system (taste in tongue and nose)+Olfactory system (aroma from orthonasal and retronasal smell, and tongue)+somatogsensory system (feeling in the mouth and nose).

According to airflow experiments on human cadavers, it showed that orthonasal and retronasal air moves through the nose in different ways. The nostril is of much narrower diameter than the choana, orthonasal air tends to flow in a laminar path across the nasal cavity during diaphragmatic breathing, while retronasal air takes on turbulent flow that carries more airborne substances to all parts of the olfactory epithelium. Turbulence is caused as air passes through the large aperture of the choana and backs up behind the smaller nostril. With the loss of transverse lamina, both orthonasal and retronasal air currents reach more of the olfactory epithelium, with the latter carrying the additional information liberated by mastication.

Unlike the conventional understanding that smell perceived by human being is created by the volatile substances with small molecules that volatilize (evaporate) from the liquids and foods released from within our mouths, it could be sensed by either sniffing-in by orthonasal olfacotory and or breathing-out by retronasal olfactory. The inventor hypothesizes that the smell is created by aerosol not only rich in volatile substances, but also includes particles and droplets containing less volatile and non-volatile substances.

Unlike the conventional thinking such as, mouthfeel is perceived by receptors in the epithelial cells of the oral cavity that are associated with four different types of somatosensory nerve ends. The inventor hypothesizes, aerosol formed from food and beverage as host of different airborne particles, droplets, and volatile substances are transported into retronasal channel once swallowed, and sensed by big surface area of nasal mucosa to create nose-feeling including nose-fullfeelingness. There are somatosensory fibers in the nose. They are thin nerve fibers with receptors in their terminals in the membrane lining the nasal cavity. They could be activated by certain kinds of volatile chemicals, by a rapid rush of air through the nasal cavity when sniffing; by physical properities such as those of carbonated drinks; and by moderate to high concentration of many kinds of smell molecules. So except for very quiet breathing, somatosensation is always present when we smell. The same for retronasal olfactory, airborne substances in aerosol plays an important role to activate the more sensitive somatosensory fibers in the nose when breathing-out.

Unlike the conventional understanding of taste such sweetness, bitterness, aftertaste and sour sensed by taste buds in tongue of oral cavity. The inventor hypothesizes, aerosol formed from food and beverage as host of airborne particles and droplets containing the taste substances such as sweeteners and acids are transported into retronasal channel once swallowed and sensed by the D-protein couple receptors in the olfactory bulbs. Bitterness and aftertaste eventually should not be credited totally to the tongue in mouth, but also these are a kind of orthonasal and retronasal smell.

Unlike the conventional understanding of aroma or smell resulted by the easily volatile substances sensed by either orthonasal and or retronasal olfactory. The inventor hypothesizes, aerosol formed from food and beverage hosting of airborne particle, droplets which contains also the non-volatile substances, less volatile substances play the big role for overall smell perception. All the airborne substances in the aerosols when breathing out like air-stream could activate both somatosensory receptors in additional to the smell receptors, thus enhance the overall flavor perception. There are a lot of trace amount of substances included non-detectable volatile substances in aerosol which play the big role for contribution of general impression of aroma.

Unlike the conventional understanding of sweetener such as sugar only sensed by taste buds, the inventor hypothesizes, sugars such as sucrose, especially less purified sucrose comprises good amount of volatile and non-volatile substances contribute to the sweet taste significantly. Aerosol containing these compounds could be formed from food and beverage in mouth and sensed by retronasal olfactory which enhance the sweet taste and mouthfeel of sucrose.

To explore more about where the sensors for taste, feeling, and smell are, and how these senses are created, the inventor correct that airborne substances in aerosol formed from food and beverage created in mouth play a significantly function in perceiving overall taste, rather than the erroneously traditional concept. It means orthonasal olfactory should not take full credit for smell; tongue should not take full credit for taste, oral cavity should not take full credit for mouthfeeling including the fullfeelingness. The tiny particles, the droplets, the less volatile and non-volatile substances in aerosol matter.

Without limitation of theory and hypothesis, current existing instruments in the market, such as gas chromatography (GC), gas chromatography-olfactometry (GC-O), gas chromatography-mass spectrometry (GC-MS) or gas chromatography-olfactometry-mass spectrometry (GC-O-MS) used to evaluate the smell could not represent the overall substances transported to the nose either orthonasal and or retronasal olfactory, because aerosols comprising the particles, droplets contain less volatile substances, non-volatile substances are either filtered or blocked during analysis and could not be tested by these instruments. Therefore, there is a need to develop a new method, new device to detect these aerosols occurred in food and beverage, which could provide significant help to understand and design the food and beverage.

In view of the foregoing, the inventors of the present application have addressed the above-described needs and provide a sample testing device, system and method for interrogating the relationship between chemical species in a liquid or solid composition and their impact on taste and olfactory integration.

One aspect of the present application relates to a device for retronasal and/or orthonasal testing and areosolized liquid or solid compositions. In some embodiment, the device comprises an aerosol generator capable of generating aerosol from a test sample, such as a food product or a beverage. The aerosol sampling could be connected to MS instrument to analyze its composition directly, and or it could be branched to a tube which human being could inhale or breath-in by mouth conveniently to get direct overall feeling of flavor. An embodiment of method, and or device comprises a step of connecting MS instrument and or a tube to the aerosol sampling.

The aerosol sampling could be connected to GC, GC-O, GC-MS, GC-O-MS with or without filtration for analyzing the volatile substances. An embodiment of a method and or device comprises a step of connecting aerosol sampling to one of instrument selected from GC, GC-O, GC-MS, GC-O-MS.

The aerosol samples could be cooled down and connected to HPLC, LC, HPLC-MS with or without filtration for analyzing the non-volatile substances. An embodiment of a method or device comprises a step of connecting aerosol sampling to one of instrument selected from LC, HPLC, LC-MS, HPLC-MS.

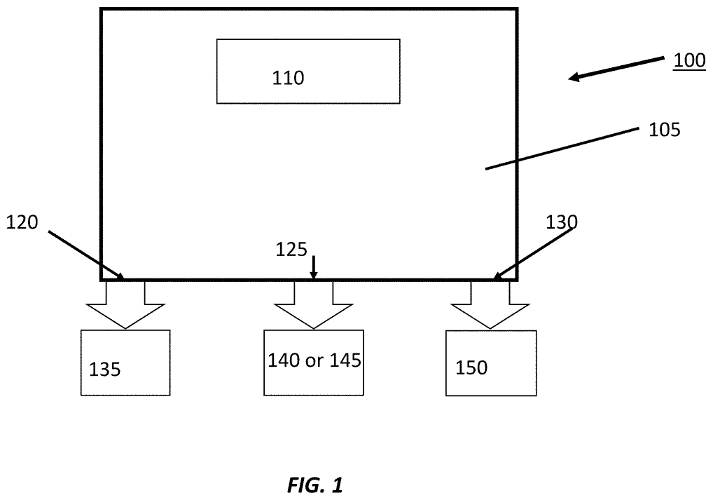

FIG. 1 depicts an exemplary sample testing device 100 that includes: an aerosol generator 105 for volatilizing a liquid or solid composition; a first port 120 for delivering a composition volatilized in the aerosol generator 105 to a retronasal passageway 135 of a subject; and/or a second port 125 for delivering the composition volatilized in the vaporization chamber 115 to a sample collection tube 140 or a sample analysis device 145. A sample testing system 160 may include the testing device 100 in combination with or the sample analysis device 145.

In certain optional embodiments further described below, the sample testing device 100 in FIG. 1 may further include a third port 130 for delivering the volatilized composition to an orthonasal passageway 150 of a subject via one or both nostrils.

In some embodiments, the aerosol generator 105 comprises an atomizer 110 that “atomizes” a liquid or solid composition, creating tiny liquid droplets that can be inhaled. Atomizer components may include one or more electronic coils wrapped by a wick.

In one embodiment, the atomizer 110 is configured for volatilizing a liquid composition. In some embodiments, the wick is made from cotton or silicon fibers receiving e.g., a liquid composition that seeps onto the fiber wrapped coil where it is heated to a temperature sufficient to vaporize the liquid, creating an aerosol mist that can be inhaled. Additional components of the aerosol generator 105 may include a power source such as a battery (e.g., by a rechargeable lithium-ion (Li-ion) battery), a chamber (or tank) for holding a liquid (e-liquid) or solid composition to be vaporized, a mouthpiece through which the subject can draw the aerosol, mist or vapor for inhalation, and control circuitry operable to actuate the vaporization component responsive to an actuation signal from a switch operative by a subject when the subject draws air through the mouthpiece by inhaling.

An atomizer 110 further includes a wire which is connected to a circuit board as well as the power supply. The circuit board (when activated by a push of a button) sends power to two places. The first is a hearing element (e.g., a thin wire coil) and the second is micro-pump located within the atomizer 110. The pump forces a liquid through the atomizer 110 where the heating element is located. As the liquid passes this it is vaporized. This process may continue until the user stops sucking or the button is released.

In one embodiment, a liquid composition is dripped into the atomizer 110 directly onto a coil (i.e., direct dripping), typically 3-15 drops, and does not involves the use of a cartridge or tank.

In some embodiments, the atomizer 110 may be configured as a cartomizer or cart. A cartomizer is typically in the form of a disposable cartridge having a built-in atomizer 110 that is prefilled with a liquid composition for vaporization and analysis. Instead of using a silica wick, a cartomizer typically utilizes fillers or Poly-fil, a cotton-like fiber used to hold a liquid composition for analysis.

In another embodiment, the atomizer 110 is configured as a clearomizer. A clearomizer is a type of atomizer 110 having a clear tank that can accommodate a larger capacity of vaporizable material and can allow one to see how much sample is left in the chamber or tank.

In another embodiment, the atomizer 110 is configured for volatilizing a solid composition. Atomizers of this class are also known in the art as “dry herb” atomizers and are used interchangeably herein with the term “solid material atomizers.” These atomizers employ a conduction heating-style in which plant materials come in direct contact with a heating element, usually ceramic or stainless steel. Temperature-wise, plant materials, including herbs and leaves begin to combust at around 400° F.; therefore, the vaporization conditions should be adjusted to operate just below that temperature. The dry herb atomizer 110 may be made from ceramic or other materials suitable for maintaining temperatures sufficient for vaporization.

A solid material atomizer 110 may include a 510-threaded connector for attachment to any 510 threaded device, such as a battery-operated power source. Compared to liquid compositions, dried plant materials may take significantly more heat to vaporize. When vaporizing such materials, however, it is preferable to ensure that the materials are not heated beyond the point of combustion.

When using an atomizer 110 configured for solid materials, it is preferable to grind the solid materials, including botanical plant materials from e.g., Stevia rebaudiana, Rubus suavissimus, Siraitia grosvenorii etc. to a fine powders, which are fully dried.

In some embodiments, the testing device 100 further includes means for delivering a volatilized composition from the aerosol generator 105 to the retronasal passageway 135 of a subject. Typically, this can be accomplished by inhaling the volatilized composition. In some embodiments, the means for delivering the volatilized composition includes means for delivering a predetermined amount of the volatilized composition to the retronasal passageway 135 of a subject.

In one embodiment, the means for delivering the volatilized composition to the retronasal passageway 135 includes the use of a tube integrally linked to the first port 120. In another embodiment, the tube is connectively linked to a disposable mouthpiece connected to the tube. In another embodiment, the means for delivering the volatilized composition to the retronasal passageway 135 includes the use of a detachable tube connectively linked to the first port 120.

In another embodiment, the present application provides means for delivering the volatilized composition to a sample collection tube 140. In some embodiment, the means for delivering the volatilized composition includes means for delivering a predetermined amount of the volatilized composition to the sample collection tube 140.

In another embodiment, the present application provides means for delivering the volatilized composition to a sample analysis device 145, including means for delivering a predetermined amount of the volatilized composition to the sample analysis device 145.

In another embodiment, the testing device 100 further includes a third port 130 for delivering the volatilized composition to an orthonasal passageway 150 of a subject.

In one embodiment, the testing device 100 includes a disposable connector containing a first tube providing a first flow path to the vaporization chamber 115 and a second tube providing a second flow path to the vaporization chamber 115, where the first tube is configured for retronasal delivery of a volatilized composition from the first port 120 into the mouth of a subject, and the second tube is configured for orthonasal delivery of the volatilized composition through the third port 130 into one or more nostrils of the subject. In a more particular embodiment, the first flow path and the second flow path merge together at a junction converging into a common flow path feeding into the vaporization chamber 115. Alternatively, the first and second flow paths may form segregated flow paths, each independently feeding into a separate vaporization chamber 115, or each feeding into a common vaporization chamber 115.

In another aspect, a sample testing system 160 includes a sample testing device 100 according to the present disclosure, where the second port 125 is connectively linked to a sample analysis device 145, such as a gas chromatography/mass spectrometry (GC-MS) device. The most common type of mass spectrometer (MS) associated with a gas chromatograph (GC) is the quadrupole mass spectrometer, sometimes referred to by the Hewlett-Packard (now Agilent) trade name “Mass Selective Detector” (MSD). Another relatively common detector is the ion trap mass spectrometer or ion trap MSn where n indicates the number mass spectrometry stages. Additionally, a magnetic sector mass spectrometer may be used. Other detectors include time of flight (TOF), single quadrupoles, tandem quadrupoles (MS-MS), triple quadrupoles, and high resolution TOF-MS:

When a second phase of mass fragmentation is added, for example using a second quadrupole in a quadrupole instrument, it is called tandem MS (MS/MS). Gas chromatography/tandem mass spectrometry (GC-MS/MS) can be used to quantitate low levels of target compounds in the presence of a high sample matrix background. Variations of the foregoing for use with the present application include gas chromatography/high resolution mass spectrometry (GC-HRMS), ultrahigh resolution mass spectrometry (Fourier Transform Ion Cyclotron Resonance Mass Spectrometer (FT-ICR-MS), and gas chromatography/time of flight mass spectrometry (GC-TOF-MS) in which an ion's mass-to-charge ratio is determined via a time of flight measurements where ions are accelerated by an electric field of known strength.

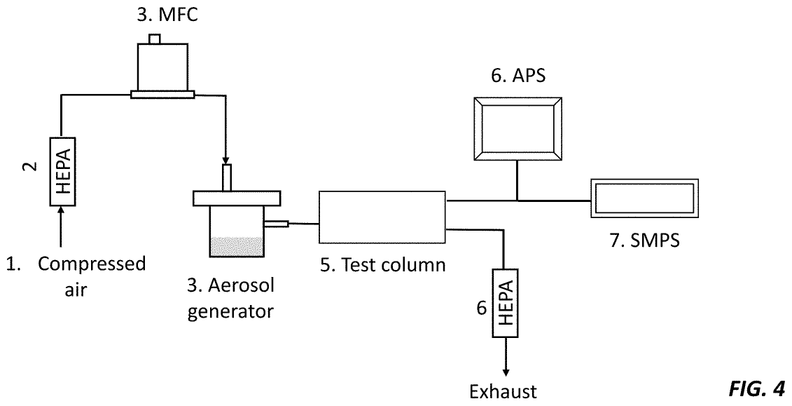

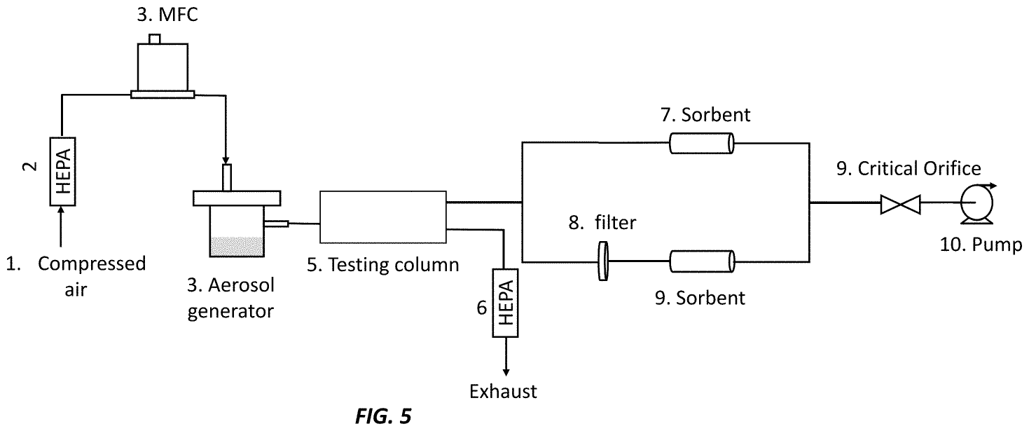

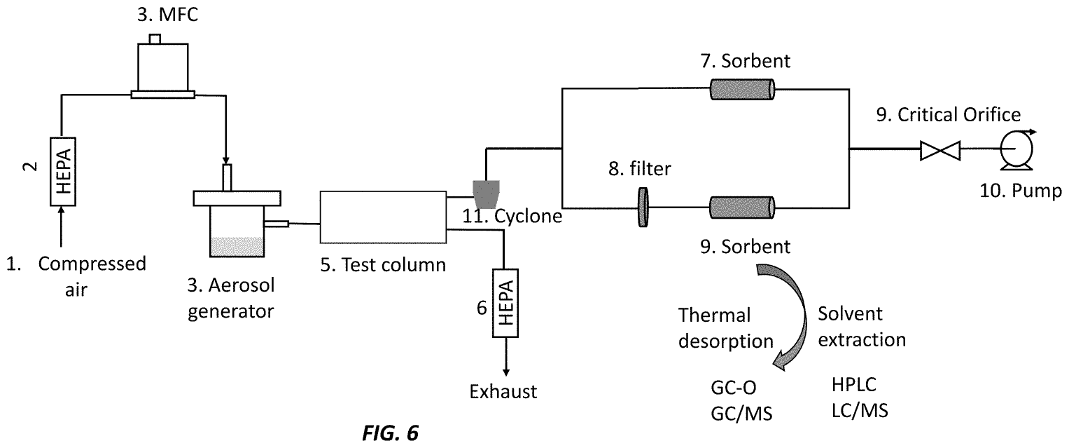

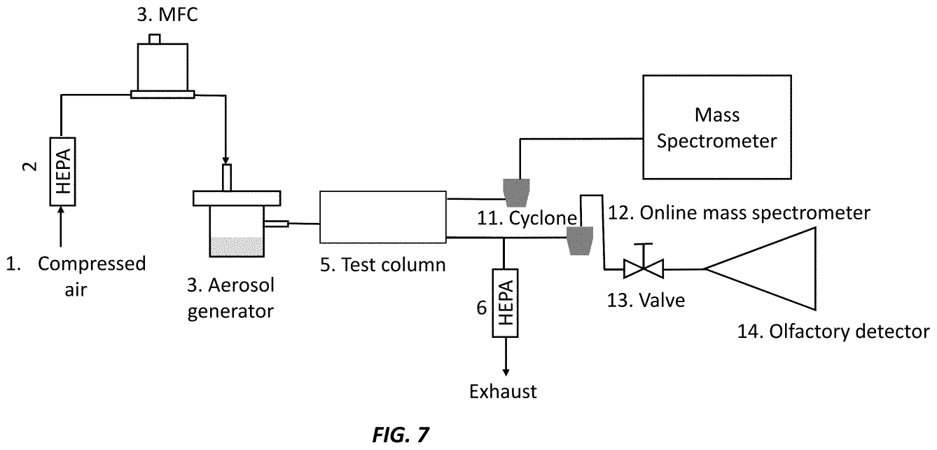

The sample analysis device 145 allows for a determination of the chemical species present in the volatilized composition and facilitates a further evaluation of chemical species associated with particular olfactory taste attributes identified by subjects utilizing the testing device 100. Additional embodiments of the device of the present application are shown in FIGS. 4-7.

Another aspect of the present application relates to a method for retronasal and/or orthonasal testing and areosolized liquid or solid compositions. In some embodiments, the method comprises the step of (1) collecting sample from one or more of sources selected from breathing-out air from human being, air created by artificial mouth during mastication or swallowing, and air or aerosol from food and beverage itself or created by atomizer, (2) determining the overall flavor the collected sample by orthonasal and/or retronasal testing, and (3) analyzing the chemical composition of the collected sample.

In some embodiments, the collected sample is an aerosol sample generated from a food or a beverage. In some embodiments, the method further comprises the step of analyzing the particle size and distribution of aerosols created from food and beverage.

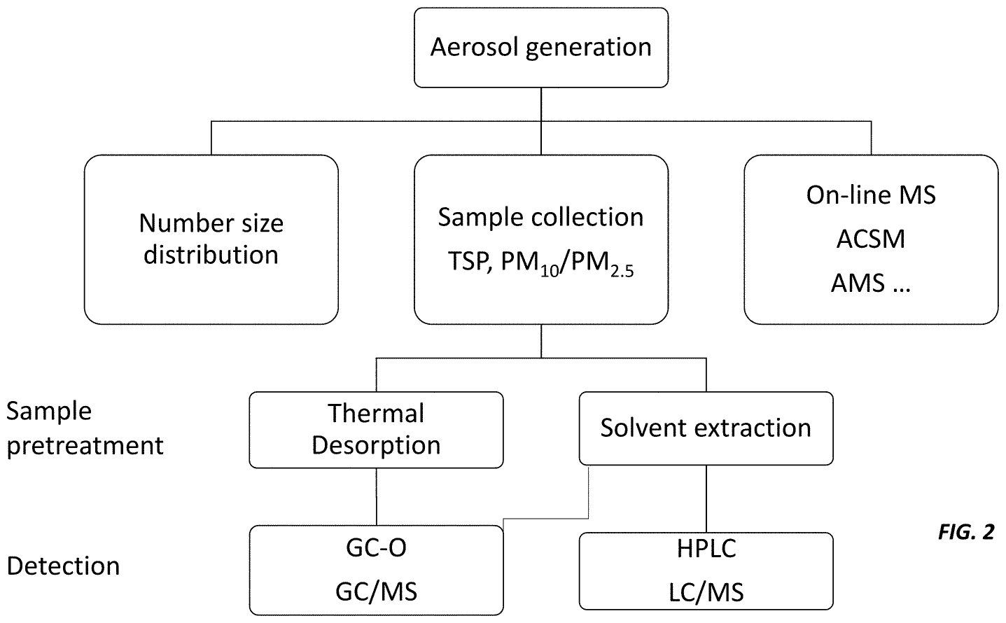

FIG. 2 is a flow diagram of an embodiment of the detection method of the present application. The method comprises the steps of generating aerosol sample from a test sample, collecting the aerosol sample and detecting the chemical composition of the aerosol sample. In some embodiments, the method further comprised the step of subjecting the aerosol sample to flavor testing. In some embodiments, the method further comprised the step of determining the size distribution and concentration of the particles in the aerosol sample. In some embodiments, the method further comprises the step of subjecting the aerosol sample to online MS analysis. In some embodiments, the method further comprises the step of pretreating the aerosol sample prior to the detecting step

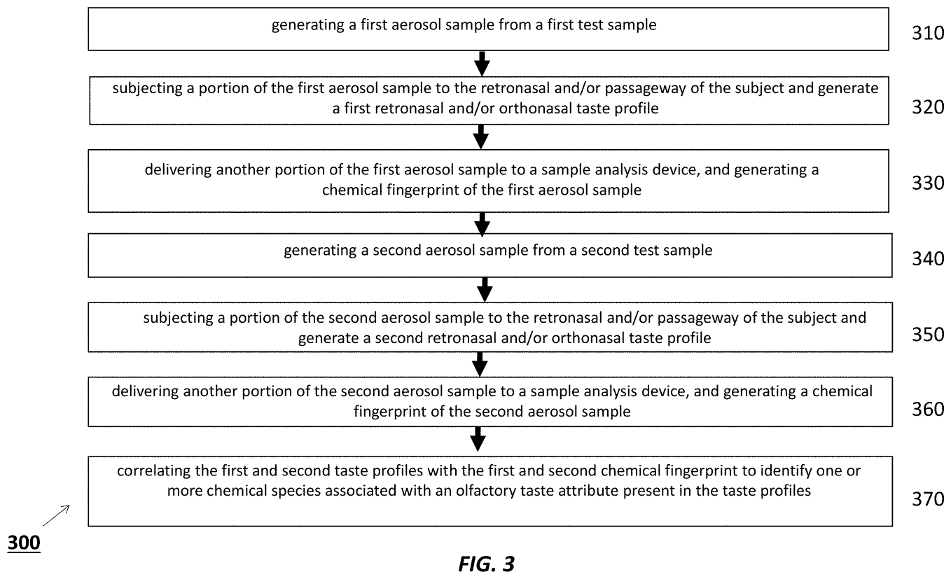

FIG. 3 is a flow diagram showing another embodiment of the method of the present application. In this embodiment, the method 300 comprises the steps of:

(a) generating (310) a first aerosol sample from a first test sample;

(b) subjecting (320) a portion of the first aerosol sample to the retronasal and/or passageway of the subject and generate a first retronasal and/or orthonasal taste profile;

(c) delivering (330) another portion of the first aerosol sample to a sample analysis device, and generating a chemical fingerprint of the first aerosol sample;

(d) generating (340) a second aerosol sample from a second test sample;

(e) subjecting (350) a portion of the second aerosol sample to the retronasal and/or passageway of the subject and generate a second retronasal and/or orthonasal taste profile;

(f) delivering (360) another portion of the second aerosol sample to a sample analysis device, and generating a chemical fingerprint of the second aerosol sample; and

(g) correlating (370) the first and second taste profiles with the first and second chemical fingerprint to identify one or more chemical species associated with an olfactory taste attribute present in the taste profiles.

In some embodiments, the methods of the present application may include the use of any of the above-described testing devices 100. As such, the method (and testing device 100) may include means for generating an aerosol sample from a liquid composition, a solid composition, or both. In one embodiment, the means for generating an aerosol sample from a composition includes actuating a testing device 100 where the device includes an atomizer 110. Alternatively, a cartridge may be inserted into the testing device 100 where the cartridge includes the atomizer(s) 110, which are activated by actuating the sample testing device 100.

In some embodiments, the method further includes the step of delivering a portion of the first and/or second aerosol sample to the retronasal passageway 135 of an evaulator. In one embodiment, the method of delivery includes the use of a tube integrally linked to a first port 120 in the sample testing device 100. In some embodiments, the tube may be connectively linked to a disposable mouthpiece. In other embodiments, the method of delivery includes the use of a detachable tube connectively linked to the first port 120. In some embodiment, the method of delivery includes means for delivering a predetermined amount of the first and/or second aerosol sample to the retronasal passageway 135.

In some embodiments, the means for delivering the first and/or second aerosol sample includes connectively linking a sample collection tube or sample analysis device 145 to the second port 125 of the sample testing device 100. In one embodiment, the step of delivering a portion of the first and/or second aerosol sample to a sample collection tube, is followed by delivery of the first and/or second aerosol sample (and/or predetermined amount thereof) in the sample collection tube to a sample analysis device 145, such as a GC-O-MS device.

In another embodiment, the step of delivering a portion of the first and/or second aerosol sample to the sample analysis device 145 includes connectively linking the second port 125 of the sample testing device 100 to the sample analysis device 145 and directly delivering the first and/or second aerosol sample (and/or predetermined amount thereof) to the sample analysis device 145.

In some embodiments, the method includes the use of a testing device 100 further comprising a third port 130 for delivering the first and/or second aerosol sample to the orthonasal passageway 150 of a subject.

In some embodiments, the method includes the use of a testing device 100 containing a disposable connector including a first tube providing a first flow path to the aerosol generator 105 and a second tube providing a second flow path to the aerosol generator 105, where the first tube is configured for retronasal delivery of a portion of an aerosol sample from the first port 120 into the mouth of a subject, and where the second tube is configured for orthonasal delivery of a portion of the same aerosol sample through the third port 130 into one or more nostrils of the subject.

In some embodiments, the method further includes the step of delivering a portion of an aerosol sample to the orthonasal passageway 150. In one embodiment, a portion of an aerosol sample is delivered to the orthonasal passageway 150 before delivery to the retronasal passageway 135. In another embodiment, a portion of an aerosol sample is delivered to the orthonasal passageway 150 after delivery to the retronasal passageway 135. Alternatively, a portion of an aerosol sample may be delivered to the retronasal 135 and orthonasal 150 passageways at the same time.

In one embodiment, a portion of an aerosol sample is delivered to the orthonasal passageway 150 through a tube integrally linked to the third port 130. In another embodiment, the tube is connectively linked to a disposable nosepiece. Alternatively, a portion of an aerosol sample may be delivered to the orthonasal passageway 150 through a detachable tube connectively linked to the third port 130.

In some embodiments, the device and methods of the present application are used for flavor evaluation for a range of compositions. The compositions may comprise one or more Stevia sweeteners, non-Stevia sweeteners, or a combination thereof. The Stevia glycoside sweeteners may include Stevia extracts, glycosylated Stevia extracts, steviol glycosides, glycosylated steviol glycosides, or combinations thereof. The non-Stevia glycoside sweeteners may include sweet tea extracts, glycosylated sweet tea extracts, rubusosides, suaviosides, glycosylated rubusosides, glycosylated suaviosides, swingle extracts, glycosylated swingle extracts, mogrosides, glycosylated mogrosides, glycyrrhizine, glycosylated glycyrrhizine, sucralose, or combinations thereof.

In some embodiments, the compositions may comprise one or more Maillard reaction products (MRPs) formed from: (1) one or more one or more Stevia glycoside sweeteners, one or more non-Stevia glycoside sweeteners, and/or one or more reducing sugars comprising a free carbonyl group; and (2) one or more amine donors comprising a free amino group.

The above description is for the purpose of teaching a person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A sample testing device, comprising:
an aerosol generator for generating aerosol from a test sample;
a sample collector for collecting aerosol particles within a desired size range;
an sample analyzer for determining chemical composition of the collected aerosol particles.

2. The testing device of claim 1, further comprising:
a first port for delivering the collected aerosol particles to a retronasal passageway of a subject; and
a second port for delivering the collected aerosol particles to the sample analyzer.

3. The testing device of claim 1, wherein the aerosol generator comprises an atomizer.

4. A method for evaluating and analyzing flavor, comprises the steps of
generating aerosol sample from a test sample;
collecting the aerosol sample;
evaluating the flavor of the collected aerosol sample; and
detecting the chemical composition of the collected aerosol sample.

5. The method of claim 4, further comprising the step of determining the size distribution and concentration of the particles in the aerosol sample.

6. The method of claim 4, further comprising the step of pretreating the aerosol sample prior to the detecting step.

7. A method for identifying chemical species associated with an olfactory taste attribute, comprising:
(a) generating a first aerosol sample from a first test sample;
(b) subjecting a portion of the first aerosol sample to the retronasal and/or passageway of the subject and generate a first retronasal and/or orthonasal taste profile;
(c) delivering another portion of the first aerosol sample to a sample analysis device, and generating a chemical fingerprint of the first aerosol sample;
(d) generating a second aerosol sample from a second test sample;
(e) subjecting a portion of the second aerosol sample to the retronasal and/or passageway of the subject and generate a second retronasal and/or orthonasal taste profile;
(f) delivering another portion of the second aerosol sample to a sample analysis device, and generating a chemical fingerprint of the second aerosol sample; and
(g) correlating the first and second taste profiles with the first and second chemical fingerprint to identify one or more chemical species associated with an olfactory taste attribute present in the taste profiles.
